(19) United States
(12) Patent Application Publication
(10) Pub. No.: US 2025/0339116 A1
(43) Pub. Date: Nov. 6, 2025

(54) G-ARM FLUOROSCOPIC MOBILE SYSTEM

(71) Applicant: BOARD OF REGENTS, THE UNIVERSITY OF TEXAS SYSTEM, Austin, TX (US)

(72) Inventor: Jorge E. LOPERA, Austin, TX (US)

(21) Appl. No.: 18/880,104

(22) PCT Filed: Jun. 30, 2023
(86) PCT No.: PCT/US2023/026717

§ 371 Date: Dec. 30, 2024

Related U.S. Application Data
(60) Provisional application No. 63/358,417, filed on Jul. 5, 2022.

(51) Int. Cl. A61B 6/00

(52) U.S. Cl. CPC A61B 6/4435; A61B 6/4405; A61B 6/487; A61B 2560/0437

(57) ABSTRACT

Various implementations include a fluoroscopic mobile device for use with a patient disposed on a support apparatus. The device includes a base, a support arm, an x-ray tube, and an image intensifier. The support arm extends from the base, the support arm comprising a first arm portion and a second arm portion. Each of the first arm portion and the second arm portion has a proximal end proximal to the base and a distal end opposite the proximal end. The x-ray tube is coupled to the distal end of the first arm portion. The image intensifier is coupled to the distal end of the second arm portion. The x-ray tube is configured to be disposed above the patient relative to a gravitational direction when the system is in use. The image intensifier is configured to be disposed between the patient and the support apparatus when the system is in use.

### CROSS-REFERENCE TO RELATED APPLICATIONS

The patent application claims the benefit of U.S. Provisional Application Ser. No. 63/358,417, filed Jul. 5, 2022, which application is incorporated by reference herein.

### BACKGROUND

A C-arm is an imaging scanner intensifier, deriving its name from the C-shaped arm used to connect the x-ray source and x-ray detector to one another. C-arms have radiographic capabilities, though they are used primarily for fluoroscopic intraoperative imaging during surgical, orthopedic, and emergency care procedures. The devices provide high-resolution X-ray images in real time, allowing the physician to monitor progress and immediately make any corrections.

However, current C-arm imaging devices require the transfer of the patient to a specially designed table to allow moving the C-arm into position both over and under the patient. This process creates challenges for physicians in cases where it is necessary to transfer intubated and critically ill patients from table to table, a process that is cumbersome and risky. The Intensive Care Unit (ICU) rooms in most hospitals have limited space for mobility and machinery, and moving a second table into the room is often impossible. Current C-arm design particularly does not allow for use with patient ICU beds because of the limited space beneath the beds and the fact that many beds are not radiolucent. A similar problem arises in the operating rooms when patients are being operated on tables that are not radiolucent and or do not provide the space underneath for placing the C-arm tube under the table.

Thus, there is a need for a fluoroscopy device that can be used with any table, bed, or other support apparatus.

### BRIEF DESCRIPTION OF THE DRAWINGS

Example features and implementations are disclosed in the accompanying drawings. However, the present disclosure is not limited to the precise arrangements and instrumentalities shown.

FIG. 1 shows a side view of a fluoroscopic mobile device for use with a patient disposed on a support apparatus, according to one implementation.

FIG. 2 shows a side view of the device of FIG. 1 being used with a wedge.

FIG. 3 shows a side view of a fluoroscopic mobile device for use with a patient disposed on a support apparatus, according to another implementation.

FIG. 4 shows a side view of a fluoroscopic mobile system including the device of FIG. 1 and a support spacer.

FIG. 5A shows an end view of the support spacer of FIG. 4.

FIG. 5B shows a side view of the support spacer of FIG. 4.

### DETAILED DESCRIPTION

The devices, systems, and methods disclosed herein provide for a novel G-arm system that allows the performance of fluoroscopic guided procedures wherever the patient is located, whether in the ICU, O.R. table, isolation room, etc. The G-arm is a portable design that allows for the image receptor to be placed beneath the patient directly, similarly to a portable X-ray plate. The fluoroscopy can thus be conducted in the bed of the patient with minimal movement of the patient and without the need for the patient to be displaced to another table or transported to a different unit

The G-Arm fluoroscopic mobile device allows the performance of many fluoroscopic guide procedures at the bedside of critically ill patients without the risk associated with transporting intubated and unstable patients to another unit, room, or table, as it is typically done currently. Because of the size, design, and convenience of the G-Arm, it is more efficient and convenient to perform fluoroscopic guided procedures in the room of isolation patients without the risk of contamination through other rooms or units.

Various implementations include a fluoroscopic mobile device for use with a patient disposed on a support apparatus. The device includes a base, a support arm, an x-ray tube, and an image intensifier. The base has a vertical axis configured to be parallel with a gravitational axis when the system is in use. The support arm extends from the base, the support arm comprising a first arm portion and a second arm portion. Each of the first arm portion and the second arm portion has a proximal end proximal to the base and a distal end opposite the proximal end. The x-ray tube is coupled to the distal end of the first arm portion. The image intensifier is coupled to the distal end of the second arm portion. The x-ray tube is configured to be disposed above the patient relative to a gravitational direction when the system is in use. The image intensifier is configured to be disposed between the patient and the support apparatus when the system is in use.

Various other implementations include a fluoroscopic mobile system. The system includes a fluoroscopic mobile device for use with a patient disposed on a support apparatus, as described above, and a support spacer. The support spacer includes a first surface, a second surface opposite and spaced apart from the first surface, a first side extending between the first surface and the second surface, and a second side opposite the first side. The at least one opening is defined by the first side. The at least one opening is configured such that the image intensifier can be disposed within the at least one opening when the patient is disposed on the first surface and the second surface is disposed on the support apparatus.

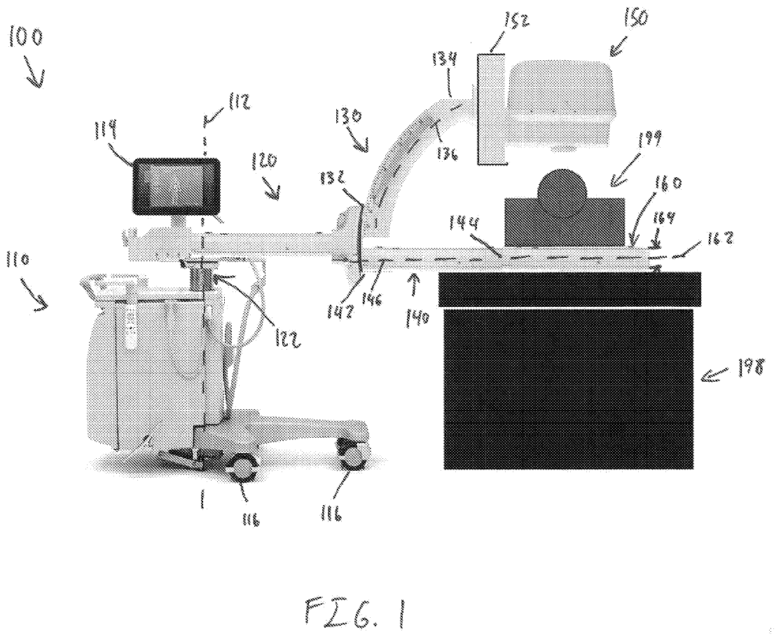

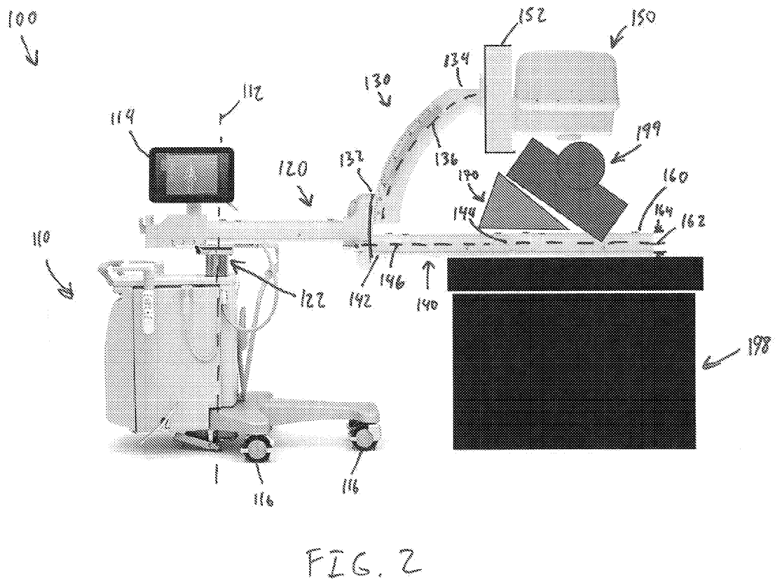

FIGS. 1 and 2 show a fluoroscopic mobile device 100 for use with a patient 199 disposed on a support apparatus 198. The device 100 includes a base 110, a support arm 120, an x-ray tube 150, and an image intensifier 160. The support apparatus 198 on which the patient 199 is disposed in FIGS. 1 and 2 is a standard hospital bed.

The base 110 of the device 100 shown in FIGS. 1 and 2 has a vertical axis 112 configured to be parallel with a gravitational axis when the system is in use in the device's 100 typical orientation. The base 110 further includes a display screen 114 and four casters 116. However, in some implementations, the base can include two or more display screens and any number of casters or no casters. The display screen(s) 114 are coupled to the main portion of the base 110 by articulating arms to allow for easier viewing.

The casters 116 allow the device 100 to be mobile such that the device 100 can be transported from one room to another room. Many existing fluoroscopy devices, such as existing C-arm devices, can only be used with special tables that allow the lower arm of the device to extend under the table for imaging. This typically involves transporting a patient to a specific room containing the special table and then moving the patient onto the special table. Because the devices disclosed herein are able to be used with any style bed or any other support apparatus, the mobility of the device can be used to move the device, rather than the patient, between different rooms.

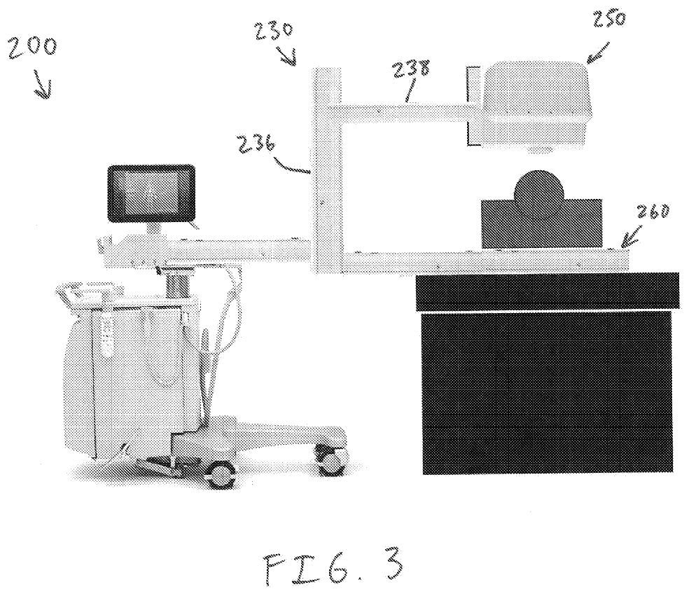

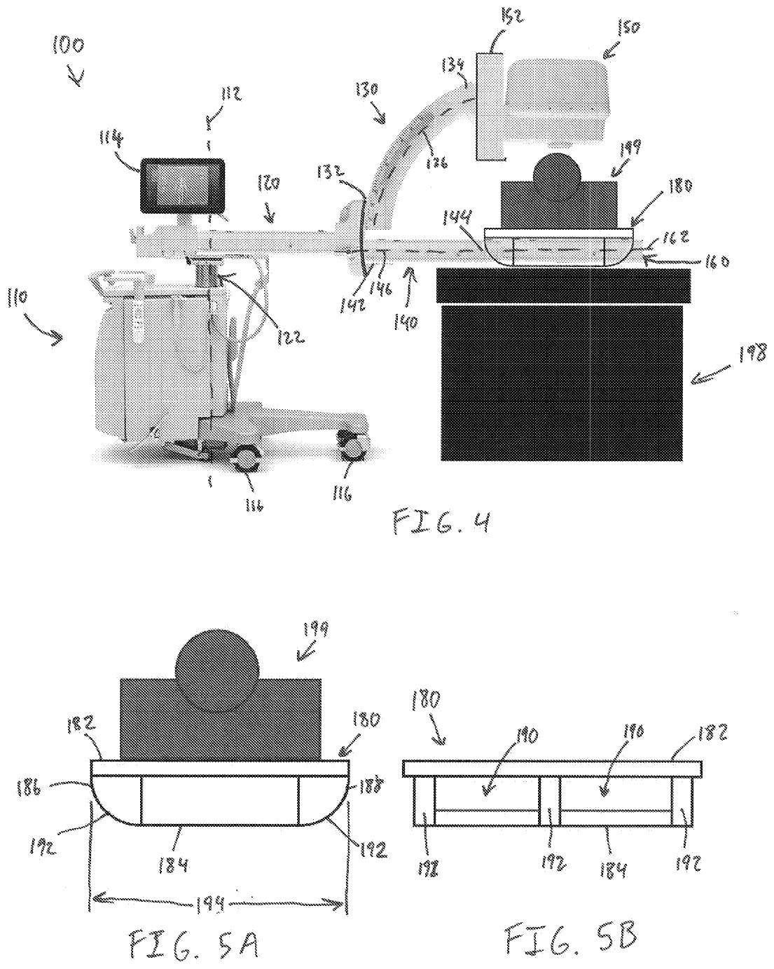

The support arm 120 extends horizontally from the base 110 and includes a first arm portion 130 and a second arm portion 140. The first arm portion 130 has a proximal end 132 proximal to the base 110 and a distal end 134 opposite the proximal end 132. The second arm portion 140 also has a proximal end 142 proximal to the base 110 and a distal end 144 opposite the proximal end 142. The first arm portion 140 extends vertically relative to the gravitational axis as it extends horizontally such that the distal end 134 of the first arm portion 130 is disposed above the distal end 144 of the second arm portion 140 relative to the gravitational axis. The first arm portion 130 shown in FIGS. 1 and 2 extends along a curved center line 136. However, in other implementations, such as the implementation shown in FIGS. 3 and 4, the first arm portion can include a vertical extension and a horizontal extension.

The x-ray tube 150 is coupled to the distal end 134 of the first arm portion 130, and the image intensifier 160 is coupled to the distal end 144 of the second arm portion 140. Because the distal end 134 of the first arm portion 130 is disposed above the distal end 144 of the second arm portion 140, the x-ray tube 150 is configured to be disposed above the patient 199 relative to a gravitational direction when the system is in use.

The second arm portion 140 extends perpendicular to the vertical axis 112 so that the longitudinal axis 162 of the image intensifier 160 is parallel with the longitudinal axis 146 of the second arm portion 140. The image intensifier 160 is configured to be disposed between the patient 199 and the support apparatus 198 when the system is in use without the image intensifier 160 or the second arm portion 140 interfering with the support apparatus 198. The image intensifier 160 shown in FIGS. 1 and 2 has a thickness 164 of 1 inch or less in a direction parallel to the vertical axis 112, which allows a patient 199 to be disposed on the image intensifier 160 with minimal discomfort. However, in some implementations, the image intensifier has a thickness of 2 inches or less in a direction parallel to the vertical axis. In some implementations, the image intensifier has a thickness of 4 inches or less in a direction parallel to the vertical axis. In some implementations, the image intensifier has a thickness of over 4 inches in a direction parallel to the vertical axis.

The x-ray tube 150 shown in FIGS. 1 and 2 is slidingly adjustable in a direction parallel to the vertical axis 112 relative to the first arm portion 130. Thus, the x-ray tube 150 can be adjusted to a position closer to, or further from, the image intensifier 160 to minimize scattered radiation during use. The x-ray tube 150 is slidingly coupled to the first arm portion 130 by a track and ball bearing 152 that can be locked in place. However, in other implementations, the x-ray tube 150 can be slidingly or otherwise adjustably coupled to the first arm portion 130 by any adjustment means known in the art. Although the x-ray tube 150 shown in FIGS. 1 and 2 is adjustable relative to the first arm portion 130, in other implementations, the x-ray tube and first arm portion are rigidly coupled and the first arm portion is adjustable in a direction parallel to the vertical axis relative to the second arm portion of the support arm.

In implementations in which the first arm portion 230 includes a vertical extension 236 and a horizontal extension 238, such as the device 200 shown in FIG. 4, the horizontal extension 238 of the first arm portion 230 can be slidingly coupled to the vertical extension 236 of the first arm portion 230 to allow for vertical adjustment of the x-ray tube 250 relative to the image intensifier 260. In other implementations, the vertical extension of the first arm portion could be telescoping such that the point at which the horizontal extension is coupled to the vertical extension moves vertically to move the x-ray tube vertically relative to the image intensifier.

The entire support arm 120 shown in FIGS. 1 and 2 is adjustable in a direction perpendicular to the vertical axis 112 relative to the base 110. This allows the support arm 120, x-ray tube 150, and image intensifier 160 to be movable closer to the base 110 such that the device 100 has a more compact size. This more compact size is useful for transporting the device 100 between rooms. The extendable support arm 120 also allows the support arm 120, x-ray tube 150, and image intensifier 160 to reach over any objects or interfering obstructions to reach the patient 199 and support apparatus 198 as needed. The support arm 120 shown in FIGS. 1 and 2 is telescoping such that the distance from the base 110 to the x-ray tube 150 and image intensifier 160 is adjustable. However, in other implementations, the support arm could be slidingly coupled to the base by a track and ball bearing that can be locked in place.

The entire support arm 120 shown in FIGS. 1 and 2 is also adjustable in a direction parallel to the vertical axis 112 relative to the base 110. This allows the support arm 120, x-ray tube 150, and image intensifier 160 to be movable higher or lower relative to the base 110 such that the device 100 has a more compact size. This more compact size is also useful for transporting the device 100 between rooms. The adjustable height of the support arm 120 also allows the support arm 120, x-ray tube 150, and image intensifier 160 to be raised or lowered to accommodate different support apparatus 198 heights. The support arm 120 shown in FIGS. 1 and 2 is slidingly coupled to the base 110 by a track and ball bearing 122 that can be locked in place. However, in other implementations, the support arm could be telescoping such that the height of the x-ray tube and image intensifier is adjustable.

In use, the device 100 is transported into a room containing a patient 199 on a support apparatus 198, such as a standard hospital bed. The device 100 is positioned beside the support apparatus 198 and the vertical height of the support arm 120 is adjusted such that the bottom surface of the image intensifier 160 is level with the top surface of the support apparatus 198 on which the patient 199 is disposed. The patient 199 is then rolled from a supine position to at least partially onto the patient's 199 side while remaining on the support apparatus 198. The support arm 120 can then be extended horizontally until the image intensifier 160 is disposed in a position such that the portion of the patient 199 to be imaged is disposed on the image intensifier 160 once the patient 199 is returned to the supine position. In some cases, the entire device 100 can be moved toward the support apparatus 198, rather than horizontally extending the support arm 120, to place the image intensifier 160 into the desired position. If padding or cushioning is desire over the image intensifier 160, radiolucent padding or cushioning can be placed on the top surface of the image intensifier 160 at this step. Once the patient 199 is returned to the supine position, the x-ray tube 150 can be adjusted vertically to minimize scattered radiation. The device 100 can then be used to image the patient 199.

In situations in which oblique imaging of the patient 199 is desired, a wedge 170, such as the one shown in FIG. 2, can be placed onto the image intensifier 160 prior to returning the patient 199 toward the supine position. Once the patient 199 is rolled back toward the supine position, the patient 199 will be resting on the wedge 170 such that imaging will occur at an oblique angle through the patient 199.

FIGS. 4-5B show a fluoroscopic mobile system including a device 100, as discussed above, and a support spacer 180. The support spacer 180 has a first surface 182, a second surface 184 opposite and spaced apart from the first surface 182, a first side 186 extending between the first surface 182 and the second surface 184, and a second side 188 opposite the first side 186. At least the first surface 182 of the support spacer 180 is made of a radiolucent material.

Two openings 190 are defined by the first side 186 and extend to the second side 188. Each of the openings 190 is configured such that the image intensifier 160 can be disposed within the opening 190 when the patient 199 is disposed on the first surface 182 of the support spacer 180 and the second surface 184 of the support spacer 180 is disposed on the support apparatus 198. Although the support spacer 180 shown in FIGS. 4-5B includes two openings 190, in other implementations, the support spacer includes only one opening or more than two openings. The first surface 182 of the support spacer 180 can include radiolucent padding or cushioning for the patient's comfort.

As seen in FIGS. 4 and 5A, the first side 186 and the second side 188 of the support spacer 180 include curved surfaces 192. The support spacer 180 has a width 194 as measured from the curved surface 192 on the first side 186 to the curved surface 192 on the second side 188. The width 194 of the support spacer 180 is larger at a portion closer to the first surface 182 than at a portion closer to the second portion 184. These curved surfaces 194 allow the support spacer 180 to be “rocked” to an angle relative to the top surface of the support apparatus 198 to facilitate an easier transition of the patient 199 from the top surface of the support apparatus 198 onto the first surface 182 of the support spacer 180.

In use, the device 100 is transported into a room containing a patient 199 on a support apparatus 198, such as a standard hospital bed. The device 100 is positioned beside the support apparatus 198 and the vertical height of the support arm 120 is adjusted such that the bottom surface of the image intensifier 160 is level with the top surface of the support apparatus 198 on which the patient 199 is disposed. The patient 199 is then rolled at least partially onto the patient's side while remaining on the support apparatus 198. The support spacer 180 is then placed onto the top surface of the support apparatus 198 and is “rocked” onto the curved surface 192 of the side of the support spacer 180 closest to the patient 199 such that the first surface 182 of the support spacer 180 is at an oblique angle to the top surface of the support apparatus 198. The patient 199 is then rolled back toward the supine position and pulled onto the first surface 182 of the support spacer 180. The pulling of the patient 199 can be performed manually or with the aid of a sling or other device for moving a patient 199. As the patient 199 is moved closer to the center of the first surface 182 of the support spacer 180, the centering of gravity of the patient 199 over the support spacer 180 causes the support spacer 180 to rock off of the curved surface 192 and back onto the second surface 184 of the support spacer 198.

The support arm 120 can then be extended horizontally until the image intensifier 160 is disposed in one of the openings 190 of the support spacer 180 such that the portion of the patient 199 to be imaged is disposed directly above the image intensifier 160. In some cases, the entire device 100 can be moved toward the support apparatus 198, rather than horizontally extending the support arm 120, to place the image intensifier 160 into the opening 190. The x-ray tube 150 can then be adjusted vertically to minimize scattered radiation, and the device 100 can be used to image the patient 199.

A number of example implementations are provided herein. However, it is understood that various modifications can be made without departing from the spirit and scope of the disclosure herein. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. Although the terms “comprising” and “including” have been used herein to describe various implementations, the terms “consisting essentially of” and “consisting of” can be used in place of “comprising” and “including” to provide for more specific implementations and are also disclosed.

Disclosed are materials, systems, devices, methods, compositions, and components that can be used for, can be used in conjunction with, can be used in preparation for, or are products of the disclosed methods, systems, and devices. These and other components are disclosed herein, and it is understood that when combinations, subsets, interactions, groups, etc. of these components are disclosed that while specific reference of each various individual and collective combinations and permutations of these components may not be explicitly disclosed, each is specifically contemplated and described herein. For example, if a device is disclosed and discussed each and every combination and permutation of the device are disclosed herein, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed systems or devices. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

What is claimed is:

1. A fluoroscopic mobile device for use with a patient disposed on a support apparatus, the device comprising:
a base having a vertical axis configured to be parallel with a gravitational axis when the system is in use;
a support arm extending from the base, the support arm comprising a first arm portion and a second arm portion, each of the first arm portion and the second arm portion having a proximal end proximal to the base and a distal end opposite the proximal end;
an x-ray tube coupled to the distal end of the first arm portion; and
an image intensifier coupled to the distal end of the second arm portion,
wherein the x-ray tube is configured to be disposed above the patient relative to a gravitational direction when the system is in use, and
wherein the image intensifier is configured to be disposed between the patient and the support apparatus when the system is in use.

2. The device of claim 1, wherein the image intensifier has a thickness of 4 inches or less in a direction parallel to the vertical axis.

3. The device of claim 2, wherein the image intensifier has a thickness of 2 inches or less in a direction parallel to the vertical axis.

4. The device of claim 1, wherein the second arm portion extends perpendicular to the vertical axis.

5. The device of claim 1, wherein the x-ray tube is adjustable in a direction parallel to the vertical axis.

6. The device of claim 5, wherein the x-ray tube is adjustable in the direction parallel to the vertical axis relative to the first arm portion.

7. The device of claim 1, wherein the support arm is adjustable in a direction perpendicular to the vertical axis.

8. The device of claim 7, wherein the support arm is adjustable in a direction perpendicular to the vertical axis relative to the base.

9. The device of claim 1, wherein the support arm is adjustable in a direction parallel to the vertical axis.

10. The device of claim 9, wherein the support arm is adjustable in a direction parallel to the vertical axis relative to the base.

11. A fluoroscopic mobile system, the system comprising:
a fluoroscopic mobile device for use with a patient disposed on a support apparatus, the device comprising:
a base having a vertical axis configured to be parallel with a gravitational axis when the system is in use,
a support arm extending from the base, the support arm comprising a first arm portion and a second arm portion, each of the first arm portion and the second arm portion having a proximal end proximal to the base and a distal end opposite the proximal end,
an x-ray tube coupled to the distal end of the first arm portion, and
an image intensifier coupled to the distal end of the second arm portion,
wherein the x-ray tube is configured to be disposed above the patient relative to a gravitational direction when the system is in use, and
wherein the image intensifier is configured to be disposed between the patient and the support apparatus when the system is in use; and
a support spacer comprising a first surface, a second surface opposite and spaced apart from the first surface, a first side extending between the first surface and the second surface, and a second side opposite the first side, wherein at least one opening is defined by the first side, wherein at least one opening is configured such that the image intensifier can be disposed within the at least one opening when the patient is disposed on the first surface and the second surface is disposed on the support apparatus.

12. The system of claim 11, wherein the image intensifier has a thickness of 4 inches or less in a direction parallel to the vertical axis.

13. The system of claim 12, wherein the image intensifier has a thickness of 2 inches or less in a direction parallel to the vertical axis.

14. The system of claim 11, wherein the second arm portion extends perpendicular to the vertical axis.

15. The system of claim 11, wherein the x-ray tube is adjustable in a direction parallel to the vertical axis.

16. The system of claim 15, wherein the x-ray tube is adjustable in the direction parallel to the vertical axis relative to the first arm portion.

17. The system of claim 11, wherein the support arm is adjustable in a direction perpendicular to the vertical axis.

18. The system of claim 17, wherein the support arm is adjustable in a direction perpendicular to the vertical axis relative to the base.

19. The system of claim 11, wherein the support arm is adjustable in a direction parallel to the vertical axis.

20. The system of claim 19, wherein the support arm is adjustable in a direction parallel to the vertical axis relative to the base.

21. The system of claim 11, wherein the first side of the support spacer includes at least one curved surface, wherein the support spacer has a width as measured from the at least one curved surface to the second side, wherein the width is larger at a portion closer to the first surface than at a portion closer to the second portion.

22. The system of claim 11, wherein the first surface comprises padding.
